(19) United States
(12) Patent Application Publication
(10) Pub. No.: US 2010/0227793 A1
(43) Pub. Date: Sep. 9, 2010

(54) Compositions and Methods for Treating Amyotrophic Lateral Sclerosis

(76) Inventors: Scott Thomas Brady, Chicago, IL (US); Gerardo Andres Morfini, Chicago, IL (US)

Correspondence Address: LICATA & TYRRELL P.C., 66 E. MAIN STREET, MARLTON, NJ, 08053, US

(21) Appl. No.: 12/717,452

(22) Filed: Mar. 4, 2010

Related U.S. Application Data
(60) Provisional application No. 61/157,329, filed on Mar. 4, 2009.

(51) Int. Cl. A61K 38/00; C12Q 1/34; C12N 5/00

(52) U.S. Cl. 514/2; 435/18; 435/375

(57) ABSTRACT

The invention relates to methods for preserving fast axonal transport in a cell affected by Amyotrophic Lateral Sclerosis by inhibiting pathogenic superoxide dismutase-induced increases in p38α activity. The present invention also provides methods for identifying agents which inhibit the phosphorylation of the kinesin-1, as well as methods for monitoring treatment of Amyotrophic Lateral Sclerosis based on the phosphorylation of p38a, neurofilament heavy chain subunits, and serines 175 and/or 176 of kinesin-1.

### INTRODUCTION

This application claims the benefit of U.S. Provisional Application No. 61/157,329, filed Mar. 4, 2009, which is herein incorporated by reference in its entirety.

This invention was made in the course of research sponsored by the National Institutes of Health (NIH grant Nos. NS23868, NS23320, NS41170, and NS43408). The U.S. government has certain rights in this invention.

### BACKGROUND OF THE INVENTION

Amyotrophic Lateral Sclerosis (ALS, or Lou Gehrig's disease) is a progressive, adult-onset, age-dependent, and uniformly lethal human neurodegenerative disease mainly affecting the function and survival of motor neurons (Bruijn, et al. (2004) Annu. Rev. Neurosci. 27:723). Relentlessly, muscles of ALS patients lose function as their motor neuron partners gradually degenerate. With their cognitive abilities unaffected, patients gradually become prisoners of their own bodies. ALS typically leads to death from respiratory paralysis 4-5 years after the onset of symptoms. Sadly, pathogenic mechanisms underlying ALS remain largely unknown, and consequently no effective therapeutic treatments are currently available. Most ALS cases are sporadic (sALS) with no known genetic defect, but 5-10% of ALS cases are inherited (Bruijn, et al. (2004) supra). Mutations in several genes have been identified and found to cause familial forms of ALS (fALS), including mutations in superoxide dismutase 1 (SOD1; Deng, et al. (1993) Science 261:1047), VAPB (a vesicle trafficking protein; James & Talbot (2006) Biochim. Biophys. Acta 1762(11-12):986-1000), senataxin (a helicase; James Talbot (2006) supra), and dynactin, a cytoplasmic dynein accessory protein implicated in retrograde axonal transport (Puls, et al. (2003) Nat. Genet. 33(4):455). Intriguingly, polypeptides derived from these genes appear functionally and structurally unrelated to each other. Moreover, the clinical phenotypes of sALS patients and those of patients with fALS of different genetic etiologies are barely distinguishable, suggesting that sALS and fALS forms share common pathogenic mechanisms (Bruijn, et al. (2004) supra). Identification of fALS-causing mutations has facilitated the generation of cellular and animal ALS models, and defined molecular disease mechanisms for use in the development of novel therapeutic strategies.

The most characterized etiological factor in fALS is mutations in the SOD1 gene. The protein derived from this gene corresponds to a 153-amino acid residue enzyme that exists as a homodimer in vivo (Williamson, et al. (2000) Science 288(5465):399). SOD1 is known to function in the conversion of superoxide (a cytotoxic product derived from mitochondrial oxidative phosphorylation) to water or hydrogen peroxide (Williamson, et al. (2000) supra). Over one hundred SOD1 mutations causing fALS have been identified and found distributed throughout the primary and quaternary structures of the protein (Bruijn, et al. (2004) supra). It is believed that a fundamental perturbation in SOD1-mediated fALS involves conformational instability of the SOD1 protein, which results in the formation of SOD1 monomers (Strange, et al. (2003) J. Mol. Biol. 328(4):877). Transgenic expression of mutant SOD1 proteins in mice triggers motor neuron disease, with pathological phenotypes closely mirroring human ALS (Price, et al. (2000) Ann. NY Acad. Sci. 920:179). Extensive analyses of mutant SOD1 transgenic mice indicated that the pathogenic effects of mutant SOD1 resulted from a toxic gain of function mechanism, but the identity of such function has not been described (Bruijn, et al. (2004) supra). Expression of mutant SOD1 disturbs multiple physiological processes in vivo. Early in the course of the disease, intrinsic pathologies of motor neurons include abnormal phosphorylation of cytoskeletal proteins (i.e., neurofilaments), instability of neuromuscular junctions, heightened electrical excitability, altered mitochondrial function, and impaired axonal transport (Bruijn, et al. (2004) supra). At later stages, non-neuronal cells such as astrocytes and microglia contribute significantly to disease progression by activating neuroinflammatory processes (Yamanaka, et al. (2008) Proc. Natl. Acad. Sci. USA 105(21):7594; Boillee, et al. (2006) Neuron 52(1):39). Contrasting with the ubiquitous tissue expression of SOD1, motor neurons are the main cell type affected in ALS, suggesting that one or more characteristics of these cells make them uniquely vulnerable to the toxic gain of function associated with SOD1 mutations (Bruijn, et al. (2004) supra).

Alterations in fast axonal transport (FAT), the cellular process involved in the translocation and delivery of membrane proteins and lipid components along axons, have been documented in neurodegenerative disorders without involvement of mutations in motor proteins. Specifically, FAT alterations were found to result from abnormal activation of specific protein kinases involved in the regulation of molecular motor proteins. Significantly, abnormal patterns of protein phosphorylation have been documented in ALS (Mizusawa, et al. (1989) Acta Neuropathol. 79(1):317; Tortarolo, et al. (2003) Mol. Cell. Neurosci. 23(2):180; Hu, et al. (2003) J. Neurochem. 85(2):422). In addition, increased activation of various protein kinases has been reported in ALS patients and fALS-related SOD1 mice (Krieger, et al. (2003) Trends Pharmacol. Sci. 24(10):535; Tortarolo, et al. (2003) supra; Hu, et al. (2003) supra; Wagey & Krieger (1998) Prog. Drug Res. 51:133; Nguyen, et al. (2001) Neuron 30(1):135). Abnormal activation of CDK5 (Nguyen, et al. (2001) supra), GSK3 (Koh, et al. (2005) Eur. J. Neurosci. 22(2):301), Protein Kinase C (PKC; Hu, et al. (2003) supra), and the mitogen-activated protein kinases (MAPKs) JNK (Hu, et al. (2003) supra) and p38 (Tortarolo, et al. (2003) supra; Hu, et al. (2003) supra; Bendotti, et al. (2004) J. Neuropathol. Exp. Neurol. 63(2):113) have all been shown in association with ALS. However, mechanisms underlying activation of these kinases and relevant pathogenic targets for these kinases have not been described.

While motor neuron cell death represents the final outcome of ALS, it is now established that these cells degenerate follow a “dying-back” pattern. Indeed, pathological observations from transgenic mice expressing SOD1 mutants and ALS patients indicate that alterations in synaptic function and axonal integrity occur much earlier than cell death (Fischer, et al. (2004) Exp. Neurol. 185(2):232). Moreover, genetic ablation of the pro-apoptotic Bax gene in transgenic mice expressing the well-characterized fALS-related SOD1 mutant G93A (G93A-SOD1) rescues motor neurons from cell death (Gould, et al. (2006) J. Neurosci. 26(34):8774). However, denervation and axonal degeneration proceeded unchanged in these animals, and no improvement in life span was observed (Gould, et al. (2006) supra). Together, these studies indicate that the molecular steps of cell death pathways constitute a late pathogenic event in ALS (Conforti, et al. (2007) Trends Neurosci. 30(4):159) that do not provide a suitable therapeutic target.

The complex functional architecture of neurons makes these cells uniquely vulnerable to even small alterations in cellular processes that fulfill their unique challenges. Supporting this idea, genetic studies have identified mutations in genes coding for microtubule-based molecular motors responsible for FAT in both humans and mice that result in dying-back neuropathies (Morfini, et al. (2005) Trends Molec. Med. 11:64). For example, loss of function mutations in selected protein subunits of conventional kinesin (Reid, et al. (2002) Am. J. Hum. Genet. 71:1189) and cytoplasmic dynein (CDyn; Puls, et al. (2005) Ann. Neurol. 57:687; Hafezparast, et al. (2003) Science 300:808) result in motor neuron pathologies resembling ALS. These discoveries suggested that reductions in FAT might be linked to ALS pathogenesis (Reid, et al. (2002) Am. J. Hum. Genet. 71(5):1189; Fichera, et al. (2004) Neurology 63(6):1108). Accordingly, several independent studies have documented alterations in FAT as a pathogenic component of ALS. Over a decade ago, in vivo studies suggested this possibility in patients with sALS (Sasaki & Iwata (1996) Neurology 47(2):535). More recently, reductions in FAT have been documented in cellular and animal models of ALS (Sasaki, et al. (2005) Acta Neuropathol. (Berl) 110(1):48; De Vos, et al. (2007) Hum. Mol. Genet. 16(22):2720). Reduction in the anterograde transport and increased accumulation of abnormally phosphorylated neurofilaments in the proximal axon have been reported at much earlier times than the onset of symptoms in mutant SOD1 mice, before pathological changes in the spinal cord can be seen (Williamson & Cleveland (1999) Nature Neurosci. 2:50). Despite these observations, little is known about the molecular mechanisms underlying axonal degeneration in ALS, including the neuronal compartment where the primary lesion takes place (Conforti, et al. (2007) supra).

### SUMMARY OF THE INVENTION

The present invention a method for preserving fast axonal transport by contacting a cell that expresses a pathogenic superoxide dismutase 1 polypeptide with an effective amount of an agent that inhibits p38α activity thereby preserving fast axonal transport in the cell.

The present invention also provides a method for preventing or treating Amyotrophic Lateral Sclerosis by administering to a subject in need of treatment an effective amount of an agent that inhibits p38α activity thereby preventing treating the subject's Amyotrophic Lateral Sclerosis.

A method for identifying an agent for treating Amyotrophic Lateral Sclerosis is also provided. This method involves the steps of contacting p38α with a test agent in the presence of kinesin-1 or a neurofilament heavy chain subunit, or a substrate fragment thereof, and determining whether the test agent inhibits the phosphorylation of the kinesin-1, neurofilament heavy chain subunit, or substrate fragment by the p38α thereby identifying an agent for treating Amyotrophic Lateral Sclerosis.

The present invention further provides a method for monitoring treatment of Amyotrophic Lateral Sclerosis is by determining, in a biological sample from a subject receiving therapy for Amyotrophic Lateral Sclerosis, the phosphorylation state of p38α, a neurofilament heavy chain subunit, or kinesin-1, wherein a decrease in the phosphorylation of p38α, neurofilament heavy chain subunit, or kinesin-1 after receiving therapy is indicative of treatment of the Amyotrophic Lateral Sclerosis.

### BRIEF DESCRIPTION OF THE DRAWINGS

FIG. 1 shows that fALS-related mutant SOD1 polypeptides selectively inhibit anterograde, conventional kinesin-dependent FAT. Quantitation of vesicle motility assays in isolated squid axoplasm indicates that effects of mutant SOD1 on anterograde FAT are significantly different (#) from WT-SOD1-perfused axoplasm (p≦0.01). Pathogenic, fALS-related SOD1 constructs tested showed no effect on retrograde FAT. Effects of mutant SOD1 were significantly different from control axoplasms at p0.01.

FIG. 2 shows that p38 mediates the inhibition of anterograde FAT by pathogenic SOD1. The results illustrate the protective effects of SB203580 (SB), and AKA-035B (AK-B) compounds, as well as the lack of protection afforded by CREBp (C), 5P600125 (SP) and AKA-035A (AK-A) compounds. (#, significantly different from SOD1 WT). Only compounds and treatments that inhibit p38α prevent the inhibition of anterograde FAT.

FIG. 3 shows that active p38α mimics the effects of pathogenic SOD1 on anterograde FAT. The results show that p38δ had no effect on FAT, but p38γ affected anterograde FAT to a lesser extent than p38α and required a higher concentration (100 nM as opposed to 10 nM). From all p38 isoforms tested, p38α most closely mimicked the effects of pathogenic SOD1, indicating that this isoform mediates the inhibitory effects of SOD1 mutants on FAT. (#, significantly different from SOD1 WT at p≦0.01)

FIG. 4 shows that the serines 175 and 176 in kinesin-1 (underlined), which are directly phosphorylated by p38α, are conserved among squid, mice and human KHC sequences

### DETAILED DESCRIPTION OF THE INVENTION

Consistent with the early appearance of axonal defects in transgenic mutant SOD1 mice, a marked activation of p38 has been demonstrated early in the course of ALS pathogenesis. Studies in isolated squid axoplasm show that p38 is specifically activated by mutant SOD1 polypeptides in an axon-autonomous and transcription-independent manner. Moreover, activation of axonal p38 is mediated by a phosphorylation-dependent mechanism involving the activity of MKKKs and MKKs upstream of p38. Further, it has now been shown that pathogenic SOD1 polypeptides inhibit anterograde, conventional kinesin-dependent FAT by a mechanism involving the activation of axonal p38. Taken together, this analysis reveals a novel gain of function mechanism for mutant SOD1 and provides a mechanistic basis linking abnormal activation of axonal p38 to reductions in FAT and increases in neurofilament phosphorylation in ALS. These observations indicate that increased activation of p38 in motor neurons and consequent inhibition of conventional kinesin-based fast axonal transport represents a critical pathogenic event in ALS. As such, the identified correlation of pathogenic SOD1 on p38 kinase activation and fast axonal transport inhibition in ALS pathogenesis provides a novel therapeutic target to limit, delay or prevent progressive neuronal degeneration in this disease.

Accordingly, the present invention provides a method for preserving fast axonal transport in a cell that expresses a pathogenic form of SOD1 polypeptide by inhibiting p38α activation and p38α-dependent phosphorylation of kinesin-1. For the purposes of the present invention, fast axonal transport is defined as conventional kinesin and cytoplasmic dynein-mediated movement of mitochondria, lipids, synaptic vesicles, proteins, and other membrane-bound organelles and cellular components to and from a neuron's cell body through the axonal cytoplasm (the axoplasm) (Morfini, et al. (2006) In: Basic Neurochemistry (Ed. Siegel, et al.) pp. 485-502). Axonal transport is also responsible for moving molecules destined for degradation from the axon to lysosomes to be broken down. Axonal transport can be divided into anterograde and retrograde categories. Anterograde transport carries products like membrane-bound organelles, cytoskeletal elements and soluble substances away from the cell body towards the synapse and other axonal subdomains (Oztas (2003) Neuroanatomy 2:2-5). Retrograde transport sends chemical messages and endocytosis-derived products from the axon back to the cell body. In accordance with particular embodiments of the present invention, agents that inhibit p38α-dependent phosphorylation of kinesin-1 prevent the inhibition of anterograde transport induced by a pathogenic SOD1 polypeptide. In this regard, the term “preserving fast axonal transport” is intended to include restoring FAT in cells with FAT defects as well as preventing such defects from occurring.

Cells that express a pathogenic form of SOD1 polypeptide include cells, in particular neurons, of or from a subject with ALS as well as neurons of or from a model system (e.g., an animal model or cell line as disclosed herein) of ALS. In this regard, the cells can undergo pathogenesis, because of expressing the pathogenic SOD1 polypeptide or alternatively, the cells can be induced to express the pathogenic SOD1 polypeptide by recombinant approaches. Such recombinant expression of proteins in cells is conventional in the art and any suitable method and expression system can be employed. In some embodiments, cells of the present invention are isolated (e.g., grown in vitro). In other embodiments, cells of the instant method are in vivo.

The amino acid sequence of wild-type human SOD1 is found under, e.g., GENBANK Accession No. NP 000445. Pathogenic SOD1 polypeptides have been described and are well-known to those of skill in the art to be associated with ALS (Cudkowicz, et al. (1997) Ann. Neurol. 41(2):210-21; Aguirre, et al. (1999) Europ. J. Hum. Genet. 7:599-602; Andersen, et al. (1995) Nature Genet. 10:61-66; Orrel, et al. (1997) J. Neurol. Sci. 153:46-49; Belleroche, et al. (1995) J. Med. Genet. 32:841-847; Rosen, et al. (1993) Nature 362:59-62; Deng, et al. (1993) Science 261:1047-1051). Pathogenic forms of SOD1 polypeptides include, but are not limited to ALA4VAL, ALA4THR, GLY12ARG, GLY16SER, GLY37ARG, CYS6PHE, GLU21LYS, LEU38VAL, GLY41ASP, GLY41SER, HIS43ARG, PHE45CYS, HIS46ARG, GLY72SER, HIS80ALA, LEU84VAL, GLY85ARG, ASP90ALA, GLY93CYS, GLY93ALA, GLY93AR, ASP96ASN, GLU100GLY, ILE104PHE, LEU106VAL, ILE113THR, SER134AS, LEU144SER, ALA145THR, THR1511LE, and IVS4AS, which is a T-to-G transversion in intron 4 of the SOD1 gene. In addition, modified SOD1 polypeptides with a wild-type sequence may become pathogenic in the course of disease. For example, oxidized SOD1 has been associated with sporadic ALS. In general, a pathogenic SOD1 polypeptide of the invention will exhibit an abnormal function and/or activity or an additional toxic activity or function as compared to the non-mutant or wild-type SOD1 protein. In particular embodiments, pathogenic forms of SOD1 polypeptide activate p38α thereby inhibiting anterograde FAT.

Based upon the findings disclosed herein, inhibitors of p38α, find application in blocking or inhibiting p38α activity (i.e., phosphorylation of p38α and p38α-mediated phosphorylation of neurofilament heavy chain subunits and kinesin-1) induced by pathogenic SOD1 mutant proteins, thereby preventing fast axonal transport defects elicited by pathogenic SOD1 polypeptides. Because p38α directly phosphorylates kinesin-1 and neurofilament heavy chain subunits, phosphorylation of kinesin-1 and neurofilament heavy chain subunits is said to be p38α-dependent in the context of ALS. p38α activities that can be inhibited by an agent of the invention include, e.g., any biochemical, cellular, or physiological property that results from phosphorylation of p38α and p38α enzymatic activity. An effective amount of a p38α inhibitor is an amount that measurably decreases or inhibits a property (e.g., phosphorylation) or biochemical activity possessed by the protein, e.g., kinase activity or the ability to recognize and phosphorylate a relevant substrate such as neurofilament heavy chain subunits and kinesin-1. In one embodiment, the activity of p38α is directly inhibited. In accordance with this embodiment, the inhibitory agent of the invention inhibits the transcription or translation of p38α, or alternatively inhibits the kinase activity of p38α. In another embodiment, the inhibitory agent of the invention indirectly inhibits p38α by inhibiting the MKKK-MKK-dependent activation of p38α. By inhibiting p38α kinase activity with any of these agents, phosphorylation of neurofilament heavy chain subunits and kinesin-1 by p38α is inhibited, and fast axonal transport is preserved.

Exemplary agents that inhibit the transcription or translation of p38α include, but are not limited to, ribozymes, inhibitory RNA molecules (e.g., siRNA or shRNA), antisense molecules and the like. Such molecules can be derived from the nucleotide sequence encoding p38α (e.g., as disclosed in GENBANK Accession No. NM_{—}001315) using conventional approaches. Exemplary inhibitory RNA molecules of use in the present invention include, but are not limited to, a siRNA molecule composed of p38α sense, 5′-GCG TAA TAC GAC TCA CTA TAG GCA CTA GGT GGT ACA GGG CTC-3′ (SEQ ID NO:1) and p38α antisense, 5′-GCG TAA TAC GAC TCA CTA TAG GCA GGA CTC CAT CTC TTC TTG G-3′ (SEQ ID NO:2) oligonucleotides (Makeeva, et al. (2006) Biochem J. 393(Pt 1):129-139) and the HuSH 29mer shRNA construct against MAPK14 NM_{—}001315 available from Origene Technologies, Inc. (Rockville, Md.). In particular embodiments, an agent which inhibits the transcription or translation of p38α is selective for the p38α isoform and does not inhibit the transcription or translation of p38β, p38δ or p38γ.

Exemplary agents that inhibit the kinase activity of p38α include, but are not limited to, inhibitors based on the pyrazolo-pyrimidine scaffold (Das, et al. (2008) Bioorg. Med. Chem. Lett. 18:2652-2657), indole-based heterocyclic inhibitors (Mavunkel, et al. (2003) Bioorg. Med. Chem. Lett. 13: 3087-3090), SB203580, SB202190, MWO1-2-069A-SRM (Munoz, et al. (2007) J. Neuroinflammation 4:21), MW01-2-069A and the like. While some embodiments embrace an inhibitor that inhibits more than one p38 isoform (e.g., isoform α and β), other embodiments embrace an inhibitory agent that is selective for the p38α and does not inhibit the activity of other kinases including, but not limited to p38β, p38δ or p38γ.

As indicated, other embodiments of this invention pertain to the inhibition of the MKKK-MKK-dependent activation of p38α. By inhibiting MKKK and/or MKK activity, the activation of p38α is inhibited, and hence phosphorylation of kinesin is inhibited thereby resulting in the preservation of fast axonal transport. Exemplary agents which inhibit MKKK/MKK/MAPK interactions include, but are not limited to, synthetic DVD oligopeptides (Takekawa, et al. (2005) Mol. Cell. 18(3):295-306), or synthetic CD or D peptides.

Optionally, agents that inhibit p38α-dependent phosphorylation of kinesin-1 for use in stimulating fast axonal transport and treating ALS can be identified in screening assays. In general, such screening assays include contacting a p38α with a test agent in the presence of a neurofilament heavy chain subunit and/or kinesin-1, or a substrate fragment thereof (e.g., a kinesin-1 recombinant polypeptide containing serines 175 and 176 of kinesin-1), and determining whether the test agent inhibits the phosphorylation of the neurofilament heavy chain subunit and/or kinesin-1 or substrate fragment by the p38α as compared to a control which lacks the test agent. In some embodiments, such assays are carried out in vitro. In other embodiments, such assays are carried out in vivo.

Optionally, agents that inhibit pathogenic SOD1-induced activation of p38α can be identified in screening assays. In general, such screening assays include contacting a cell or animal expressing a pathogenic form of SOD1, and determining whether the test agent inhibits the phosphorylation and activation of p38α as compared to a control or animal which lacks the test agent. In some embodiments, such assays are carried out in vitro. In other embodiments, such assays are carried out in vivo.

A kinesin of particular interest in accordance with the present invention is kinesin-1, specifically the heavy chain of conventional kinesin. Kinesin-1 (kinesin heavy chain, KIFs) is the most abundant kinesin in adult mammalian brain and is highly conserved across species. The protein sequences for kinesin-1 proteins are well-known in the art. Sequences for kinesin-1A (KIF5A) are found under GENBANK Accession Nos. NP_{—}004975 (Homo sapiens; SEQ ID NO:3), NP_{—}001034089 (Mus musculus; SEQ ID NO:4) and NP_{—}997688 ((Rattus norvegicus; SEQ ID NO:5). Sequences for kinesin-1B (KIF5B) are found under GENBANK Accession Nos. NP_{—}004512 (Homo sapiens; SEQ ID NO:6), NP_{—}032474 (Mus musculus; SEQ ID NO:7), and NP_{—}476550 (Rattus norvegicus; SEQ ID NO:8). Furthermore, sequences for kinesin-1C (KIF5C) are found under GENBANK Accession Nos. NP 004513 (Homo sapiens; SEQ ID NO:9) and NP 032475 (Mus musculus; SEQ ID NO:10). As depicted in FIG. 4, the location of phosphorylated serines 175 and 176 are highly conserved across species. Accordingly, particular embodiments embrace inhibiting the phosphorylation of serine 175 and 176 of SEQ ID NO:9 or SEQ ID NO:10, or a substrate fragment containing the same.

As with kinesin-1, neurofilament heavy chain subunits are known in the art. For example, GENBANK Accession Nos. NP_{—}066554, NP_{—}035034, and NP_{—}036739 provide the amino acid sequence of human, mouse and rat neurofilament heavy chain subunits of use in accordance with the present invention. Substrate fragments of neurofilament heavy chain subunits include those fragments capable of being phosphorylated by p38α. By way of illustration residues 518, 526 and 532 are known phosphorylation sites of human neurofilament heavy chain subunits. Accordingly, a fragment containing one or more of these residues would be a suitable substrate fragment.

According to in vitro aspects of the screening assay of the invention, a putative inhibitory agent is incubated under appropriate conditions in vitro in the presence of p38α, a p38α substrate (e.g., kinesin-1 or neurofilament heavy chain subunit, or substrate fragment thereof), and a phosphate donor (e.g., adenosine triphosphate, ATP), and it is determined whether the substrate is phosphorylated. Isolated p38α protein can be obtained for this, as well as other assays, by several different molecular and chromatographic methods known to those skilled in the art. Optionally, isolated p38α can be obtained from a commercial source such as Enzo Life Sciences International, Inc. (Plymouth Meeting, Pa.). The p38α polypeptide useful in the methods of the present invention is preferably wild-type whose sequence is known and readily available. For example, the human p38α polypeptide is available under GENBANK Accession No. NP_{—}001306. By way of illustration of the present screening assay, isolated p38α protein (˜0.5 μg to ˜2 μg) is incubated with substrate (e.g., kinesin at ˜1 μg to ˜3 μg) in an aqueous medium, such as a kinase buffer (containing, e.g., about 20 mM HEPES, pH 7.5, 15 mM MgCl₂, mM β-glycerophosphate, 0.1 mM Na₂PO₄ and 2 mM dithiothreitol) at about 30° C. for approximately 15 minutes. The phosphate donor, ATP, is added at approximately 100 μM. For detection purposes, 5 μCi of γ-³²P-ATP can be used as a co-substrate. Besides control assays, the assay system also includes one or more test agents. The reaction can be terminated by addition of Laemmeli buffer, approximately 20 μl. The addition of this buffer will also prepare the sample for product analysis. The reaction mixture can be subjected to sodium dodecylsulfate polyacrylamide gel electrophoresis (hereinafter SDS-PAGE) in order to determine the amount of phosphorylated kinesin-1 that was formed in the reaction. The radioactivity emitted from the γ-³²P can be measured using conventional radioactivity gel detection systems, such as an X-ray film autoradiography or PHOSPHORIMAGER scan. A determination can then be made concerning whether the test agent inhibits p38α activity by comparing reaction mixtures having the agent present to reaction mixtures without addition of the compound.

Alternatively, p38α substrates, such as kinesin-1 or neurofilament heavy chain subunit and ATP, can be incubated in the presence of a cellular extract containing p38α enzyme activity. An inhibitory agent to be tested can be placed in the reaction vial along with the other reactants to examine the efficacy of the agent. The reaction and detection protocol can be conducted in the same manner as that described above for the in vitro assay. The cellular extract can originate from a cell or tissue culture system, or can be prepared from whole tissue employing isolation and purification protocols known to those skilled in the art.

In another embodiment, the invention pertains to contacting a cell with a putative inhibitory agent in order to screen for inhibitory agents of p38α activity in an in vivo assay. The cell to be contacted can be of a cell or tissue culture system. If the agent is not membrane permeable, then the agent can be delivered into the cell via electroporation, or if it is a polypeptide, a nucleic acid or viral vector can be employed. If the cell lacks a p38α gene or functional p38α gene or transcript or translational product, the cell can be transfected with an operatively linked p38α gene. “Operatively linked” is intended to mean that the nucleotide sequence is linked to a regulatory sequence in a manner which allows expression of the nucleic acid sequence.

To detect the phosphorylated product (e.g., p38α or a p38α substrates kinesin-1 or neurofilament heavy chain subunit), any number of methods and protocols known to those skilled in the art can be used including, but not limited to, western blot, mass spectrometric approaches, and methods for the analysis of fast axonal transport, e.g., as disclosed herein. Antibodies, both monoclonal and polyclonal, can be made against epitopes derived from the phosphorylated site on the p38α substrate bound to a phosphate group. A SDS-PAGE procedure can be performed on homogenized cell extracts and subsequently subjected to western blot analysis using an antibody specific for a phosphorylated p38α substrate, such as kinesin-1.

In another embodiment, the invention pertains to a method for screening potential inhibitory agents of p38α activity by administering to an animal, including mammals, the agent and determining what effect, if any, the agent has on the animal's physiological status. The animal is given an amount of test agent sufficient to allow for proper pharmacodynamic absorption and tissue distribution in the animal. Preferably, the animal used is an example of a model system of ALS. However, to test the safety of the putative agent, a normal animal is preferably also subjected to the treatment. Following administration of the agent, the animal can be sacrificed and tissue sections from the brain, as well as other tissues, can be harvested and examined. In another embodiment, an animal model afflicted with ALS can be administered a p38α inhibitor and the symptoms associated with ALS are evaluated. It is contemplated that the agent can be administered before or after symptoms are evident to determine whether the agent can prevent or treat ALS. Attenuation, delay, amelioration or improvement of ALS symptoms can be assessed, whereby improvement is indicative of the inhibitors ability to prevent and/or treat ALS.

The p38α proteins useful in the methods of the invention are not limited to the naturally occurring sequences described above. p38α containing substitutions, deletions, or additions can also be used, provided that those polypeptides retain at least one activity associated with the naturally occurring polypeptide and are at least 70% identical to the naturally occurring sequence. An example of a p38α that is not naturally occurring, though useful in the methods of the invention, is a p38α-gluthathione-S-transferase (p38α-GST) fusion protein. Such a protein can be produced in large quantities in bacteria and isolated. The p38α fusion protein can then be used in an in vitro kinase assay as described herein.

Test agents encompass numerous chemical classes, although typically they are organic compounds. In some embodiments, the candidate agents are small organic compounds, i.e., those having a molecular weight of more than 50 yet less than about 2500, preferably less than about 1000 and, more preferably, less than about 500. Candidate test agents generally include functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate test agents can have a cyclic carbon or heterocyclic structure and/or aromatic or polyaromatic structures substituted with one or more of the above-identified functional groups. Candidate test agents also can be biomolecules such as peptides, proteins, antibodies, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate test agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents (e.g., those disclosed herein) can be subjected to directed or random chemical modifications such as acylation, alkylation, esterification, amidification, etc. to produce structural analogs of the agents.

A variety of other reagents also can be included in the screening assays disclosed herein. These include reagents such as salts, buffers, neutral proteins (e.g., albumin), detergents, etc. which may be used to facilitate optimal protein-protein binding. Such a reagent can also reduce non-specific or background interactions of the reaction components. Other reagents that improve the efficiency of the assay such as protease inhibitors, nuclease inhibitors, antimicrobial agents, and the like may also be used.

In particular embodiments, the agents of the present invention selectively inhibit p38α. Desirably, the p38α inhibitors selectively decrease p38α kinase activity in neurons and protect neurons by preserving fast axonal transport thereby preventing or treating ALS.

In this regard, the present invention also embraces a method for the prevention or treatment of neurological conditions, specifically ALS, either through prophylactic administration or therapeutic administration. As indicated, the presence of pathogenic SOD1 polypeptide is associated with ALS. As is conventional in the art, ALS is a neurodegenerative disorder characterized by the death of motor neurons in the brain, brainstem, and spinal cord, resulting in fatal paralysis. In so far as the clinical symptoms associated with familial and sporadic ALS indicate a common pathogenic mechanism, the present invention embraces the prevention or treatment of familial and sporadic ALS. In particular embodiments of this invention, the ALS is familial ALS, which is typically caused by mutations in the SOD1 gene that usually cause autosomal dominant disease, but can also cause autosomal recessive ALS. Alternatively, SOD1 with a normal sequence may become pathogenic due to biochemical modifications that alter SOD1 conformation and also lead to activation of p38 kinase activity in affected neurons. With regard to the appearance of symptoms and the progression of the illness, the course of the disease may include the muscle weakness in one or more of the hands, arms, legs or the muscles of speech, swallowing or breathing; twitching (fasciculation) and cramping of muscles, especially those in the hands and feet; impairment of the use of the arms and legs; “thick speech” and difficulty in projecting the voice; and in more advanced stages, shortness of breath, difficulty in breathing and swallowing.

To carry out prophylatic or therapeutic treatment, a subject in need of such treatment is administered an effective amount of an agent that inhibits p38α-dependent phosphorylation of kinesin-1 or neurofilament heavy chain subunits. An effective amount for a given agent is that amount administered to achieve the desired result, for example, the inhibition of p38α kinase activity; restoration or preservation of appropriate neurofilament heavy chain subunit or kinesin-1 phosphorylation, preservation of FAT; or attenuation, delay, amelioration of or improvement in one or more of the signs or symptoms associated with ALS. Accordingly, upon administration of a p38α inhibitor, effectiveness of treatment can be determined by measuring p38α phosphorylation or activity, or phosphorylation of kinesin-1 or neurofilament heavy chain subunits. Subjects benefiting from such treatment include those having ALS (e.g., subjects exhibiting the signs or symptoms of ALS), as well as subjects suspected of having ALS (e.g., subjects diagnosed with a pathogenic SOD1 polypeptide but not exhibiting the signs or symptoms of ALS).

Inhibitors of the present invention can be administered subcutaneously, intravenously, parenterally, intraperitoneally, intradermally, intramuscularly, topically, enteral (for example, orally), rectally, nasally, buccally, vaginally, by inhalation spray, by drug pump or via an implanted reservoir in dosage formulations containing conventional non-toxic, physiologically (or pharmaceutically) acceptable carriers or vehicles.

In a specific embodiment, it may be desirable to administer the agents of the invention locally to a localized area in need of treatment; this can be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers.

In a specific embodiment when it is desirable to direct the agent to the central nervous system, techniques which opportunistically open the blood brain barrier for a time adequate to deliver the drug there through can be used. For example, a composition of 5% mannitose and water can be used. The present invention also provides pharmaceutical compositions. Such compositions include a therapeutically (or prophylactically) effective amount of the agent, and a physiologically acceptable carrier or excipient.

Suitable pharmaceutically acceptable carriers include but are not limited to water, salt solutions (for example, NaCl), alcohols, gum arabic, vegetable oils, benzyl alcohols, polyethylene glycols, glycerol, gelatin, carbohydrates such as lactose, amylose or starch, magnesium stearate, talc, silicic acid, viscous paraffin, perfume oil, fatty acid esters, hydroxymethylcellulose, polyvinyl pyrolidone, and combinations thereof. The pharmaceutical preparations can be sterilized and if desired, mixed with auxiliary agents, for example, lubricants, preservatives, stabilizers, wetting agents, emulsifiers, salts for influencing osmotic pressure, buffers, coloring, flavoring and/or aromatic substances and the like which do not deleteriously react with the active agents.

The compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The compositions can be formulated in accordance with the routine procedure as a pharmaceutical composition adapted for intravenous administration to human beings. Typically, compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

For topical application, there are employed as nonsprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, enemas, lotions, sols, liniments, salves, aerosols, etc., which are, if desired, sterilized or mixed with auxiliary agents, for example, preservatives, stabilizers, wetting agents, buffers or salts for influencing osmotic pressure, etc. The drug may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The amount of agents which will be effective in the treatment of ALS can depend on the agent and whether the agent is being used prophylactically or therapeutically, and can be determined by standard clinical techniques. In addition, in vitro or in vivo assays can optionally be employed to help identify optimal dosage ranges. The precise dose to be employed in the formulation will also depend on the route of administration and should be decided according to the judgment of the practitioner and each subject's circumstances.

As used herein, the term “subject” is intended to include any mammal that may be in need of treatment with an agent of the invention. Subjects include but are not limited to, humans, non-human primates, cats, dogs, sheep, pigs, horses, cows, rodents such as mice, hamsters, and rats.

Having identified that phosphorylation of conventional kinesin by p38 is a primary pathogenic event in ALS, the present invention also provides to a method for monitoring or evaluating efficacy of treatment of ALS by determining, in a biological sample from a subject, the phosphorylation state of neurofilament heavy chain subunit, and/or serine 175 and/or 176 of kinesin-1 (in particular the heavy chain of kinesin-1), wherein a decrease in the amount of phosphorylated serine 175 and/or 176 of kinesin-1 as compared to an untreated sample or control sample (e.g., a sample from the subject prior to treatment) is indicative of successful or beneficial treatment of ALS. In particular embodiments, the subject is being treated with a therapeutic agent, e.g., as identified by the screening method of the invention. In another embodiment, the subject is being treated as part of a clinical trial, wherein determining the phosphorylation state of kinesin-1 is to evaluate whether a test agent is efficacious in humans.

According to the invention, a biological sample can include cells, fluids, tissues and/or organs obtained by any means such that said cells, fluids, tissues, and/or organs are suitable for determining the phosphorylation state of neurofilament heavy chain subunit and/or serine 175 and/or 176 of kinesin-1.

In some embodiments of the invention, the biological sample is presented for analysis within its native in vivo context. A non-limiting example for in vivo detection is novel magnetic resonance imaging techniques (Jacobs, et al. (2001) J. Nucl. Med. 42(3):467-475; Wunderbaldinger, et al. (2000) Eur. J. Radiol. 34(3):156-165), wherein the biological sample may be identified and subjected to analysis while remaining in a living subject throughout.

In other embodiments of the invention, the biological sample is biopsied, resected, drawn or otherwise harvested from a subject. In accordance with this embodiment, the phosphorylation state of serine 175 and/or 176 of kinesin-1 can be determined using mass spectrometry methods known in the art. Alternatively, the phosphorylation state of neurofilament heavy chain subunit and/or serine 175 and/or 176 of kinesin-1 can be determined using, e.g., an antibody which specifically recognizes the phosphorylation state of serine 175 and/or 176 of kinesin-1 (either phosphorylated or dephosphorylated). Such antibody(ies) may be delivered to cells in vitro or in vivo using particle bombardment (see, e.g., U.S. Pat. No. 5,836,905) or any other delivery technique known in the art.

An antibody is said to specifically recognize the phosphorylation state of kinesin-1 if it is able to discriminate between the unphosphorylated and phosphorylated forms of kinesin-1. For example, an antibody which specifically recognizes the phosphorylated state of kinesin-1 will only bind to kinesin-1 with a phosphorylated serine 175 and/or 176 but will not bind to kinesin-1 with an unphosphorylated serine 175 and/or 176. Conversely, an antibody that specifically recognizes the dephosphorylated state of kinesin-1 will only bind to kinesin-1 with a dephosphorylated serine 175 and/or 176 but will not bind to kinesin-1 with a phosphorylated serine 175 and/or 176.

A method of using antibodies which specifically recognize the phosphorylation state of kinesin generally involves contacting a sample with said antibody and detecting the formation of an antigen-antibody complex using an immunoassay. The kinesin-1 antigen, as used herein, includes both the phosphorylated and unphosphorylated forms, however, the phosphorylated state is preferred. The conditions and time required to form the antigen-antibody complex may vary and are dependent on the sample being tested and the method of detection being used. Once non-specific interactions are removed by, for example, washing the sample, the antigen-antibody complex is detected using any one of the well-known immunoassays used to detect and/or quantitate antigens. Exemplary immunoassays which may be used in the method of the invention include, but are not limited to, enzyme-linked immunosorbent, immunodiffusion, chemiluminescent, immunofluorescent, immunohistochemical, radioimmunoassay, agglutination, complement fixation, immunoelectrophoresis, western blots, mass spectrometry, antibody array, and immunoprecipitation assays and the like which may be performed in vitro, in vivo or in situ. Such standard techniques are well-known to those of skill in the art (see, e.g., Methods in Immunodiagnosis (1980) 2^{nd} Edition, Rose and Bigazzi, eds. John Wiley & Sons; Campbell et al. (1964) Methods and Immunology, W.A. Benjamin, Inc.; Oellerich (1984) J. Clin. Chem. Clin. Biochem. 22:895-904; Harlow and Lane (1988) Antibodies: A Laboratory Manual, Cold Spring Harbor Laboratory, New York).

Antibodies of use in accordance with the present invention can be monoclonal or polyclonal. It is contemplated that such antibodies can be natural or partially or wholly synthetically produced. All fragments or derivatives thereof which maintain the ability to specifically bind to and recognize the phosphorylation state of kinesin-1 are also contemplated. The antibodies can be a member of any immunoglobulin class, including any of the classes: IgG, IgM, IgA, IgD, and IgE. Derivatives of the IgG class, however, are preferred in the present invention.

Antibody fragments can be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment may be produced by any means. For instance, the antibody fragment may be enzymatically or chemically produced by fragmentation of an intact antibody or it may be recombinantly produced from a gene encoding the partial antibody sequence. The antibody fragment may optionally be a single-chain antibody fragment. Alternatively, the fragment may comprise multiple chains which are linked together, for instance, by disulfide linkages. The fragment may also optionally be a multi-molecular complex. A functional antibody fragment will typically comprise at least about 50 amino acids and more typically will comprise at least about 200 amino acids. As used herein, an antibody also includes bispecific and chimeric antibodies.

Naturally produced antibodies can be generated using well-known methods (see, e.g., Kohler and Milstein (1975) Nature 256:495-497; Harlow and Lane (1988) supra). Alternatively, antibodies which specifically recognize the phosphorylation state of kinesin-1 are derived by a phage display method. Methods of producing phage display antibodies are well-known in the art (e.g., Huse, et al. (1989) Science 246(4935):1275-81).

Selection of kinesin-1-specific antibodies is based on binding affinity to kinesin-1 which is either phosphorylated or unphosphorylated at serine 175 and/or 176 and can be determined by the various well-known immunoassays indicated above.

The observations presented herein provide the basis for a novel pathogenic mechanism in ALS. The data indicate that several mutant SOD1 polypeptides can activate p38 and inhibit anterograde FAT in an axon-autonomous manner. Moreover, pharmacological and biochemical data indicates that this effect of pathogenic SOD1 depends upon p38 activation. Accordingly, increased activity of this kinase was observed in G93A-SOD1 mice early in the course of disease. The findings also indicate that conventional kinesin, a major motor protein responsible for anterograde FAT, represents a novel pathogenic p38 target in ALS. Together, these observations indicate that FAT is compromised in ALS due to increased activation of axonal p38. Reductions in anterograde FAT would lead to insufficient delivery of essential cargoes to synapses and axons, and result in “dying back” degeneration of motor neurons. Therefore, the prevention of phosphorylation-dependent abnormalities derived from heightened p38 activity would ameliorate axonal degeneration in motor neurons of transgenic G93A-SOD1 mice, and ultimately in ALS patients.

The invention is described in greater detail by the following non-limiting examples.

### Example 1

### Pathogenic SOD1 Polypeptides Inhibit Anterograde Fast Axonal Transport

Although the axonal compartment represents a critical pathogenic target in ALS, axon-specific effects of mutant SOD1 have not been previously addressed. Several independent reports suggest early deficits in FAT in ALS. However, it was not clear whether those defects resulted from pathogenic events in the neuronal cell body, or from alterations of cellular processes within the axonal compartment (Conforti, et al. (2007) Trends Neurosci. 30:159-66). Therefore, axon-autonomous effects of pathogenic SOD1 on FAT were evaluated using vesicle motility assays in isolated squid axoplasm, which lacks both nuclear and protein synthetic synthesis compartments. This experimental system allows for quantitative analysis of MBOs moving in both anterograde (conventional kinesin-dependent) and retrograde (cytoplasmic dynein-dependent) FAT rates. Further, the lack of plasma membrane in this preparation facilitates an evaluation of the effects of neuropathogenic proteins on FAT.

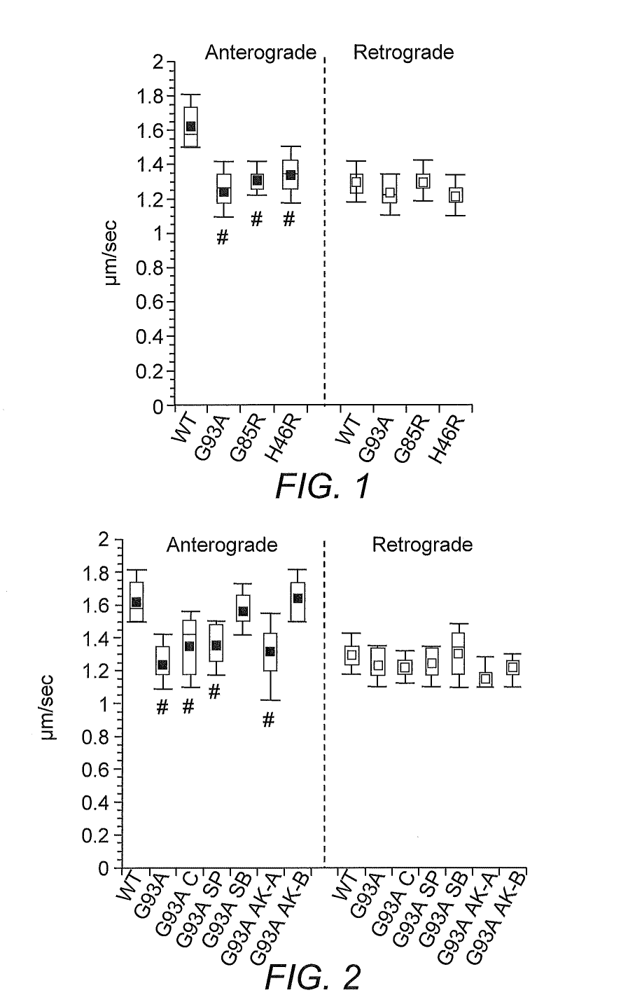

Highly purified (>95%), recombinant forms of wild-type SOD1 (WT-SOD1) and three fALS-related SOD1 mutants (G93A-SOD1, G85R-SOD1, H46R and A4V-SOD1) were according to standard methods (Strange, et al. (2003) J. Mol. Biol. 328:877-891). Polypeptides were perfused at 5 μM concentration, and both anterograde (conventional kinesin-dependent) and retrograde (CDyn-dependent) FAT rates analyzed by video-enhanced microscopy over 50 minutes. Perfusion of wild type SOD1 (WT-SOD1) had no effect on either direction of FAT. In contrast, perfusion of G93A-SOD1 selectively inhibited anterograde FAT transport rate). Similar results were obtained after perfusion of SOD1-G85R and SOD1H46R mutant variants. Quantitative analysis of these experiments indicated that all mutants SOD1 proteins tested inhibited anterograde, but nor retrograde FAT (FIG. 1). Selective inhibition of anterograde FAT by fALS-related polypeptides indicated that their effect on FAT did not result from alterations in microtubule integrity. Further, the presence of ATP (5 mM) in the perfusion buffer also ruled out mutant SOD1-induced deficits in ATP production.

Immunoprecipitation methods were used to evaluate potential interactions of conventional kinesin with mutant SOD1. Spinal cord lysates obtained from transgenic mice expressing wild-type SOD1 (WT-SOD1 mice) or G93A-SOD1 (G93A-SOD1 mice) (Gurney, et al. (1994) Science 264:1772-5) served as starting material. Conventional kinesin exists as a heterotetramer composed of two heavy chains (kinesin-1, KHC) and two light chains (KLC) (Deboer, et al. (2008) Biochemistry 47:4535-43). Antibodies recognizing kinesin-1s effectively immunoprecipitated conventional kinesin from both WT-SOD1 and G93A-SOD1 mouse spinal cord lysates. However, anti-SOD1 antibodies failed to detect SOD1 in these immunoprecipitates, indicating that the inhibitory effect of mutant SOD1 polypeptides on anterograde FAT does not result from direct interactions with this motor protein. Taken together, results from these experiments indicated that fALS-related SOD1 mutant polypeptides can inhibit conventional kinesin-based FAT in manner independent of alterations in the neuronal cell body or physical interactions between SOD1 and motor proteins.

### Example 2

### Pathogenic SOD1 Increases Neurofilament Phosphorylation

It was determined whether the molecular mechanisms underlying inhibition of anterograde FAT were induced by fALS SOD1 mutant proteins. It has been shown that several mutant polypeptides associated with familial forms of neurodegenerative diseases induce the activation of axonal kinases involved in FAT regulation. Moreover, various studies have documented abnormal activation of protein kinases in spinal cords of ALS patients and ALS mouse models.

To determine whether mutant SOD1 could induce activation of axonal kinases, metabolic labeling experiments were performed in isolated axoplasms. These procedures helped evaluate effects of WT-SOD1 and G93-SOD1 polypeptides on the phosphorylation pattern of axonal proteins. Two axons from the same squid (“sister” axons) were dissected and extruded. One axon was perfused with WT-SOD1, whereas the contralateral “sister” axon was perfused with G93A-SOD1 in the presence of P³²-radiolabelled ATP. After a 50-minute incubation, axoplasms were lysed and analyzed by autoradiography. Neurofilament (NF) proteins represent the major phosphoproteins proteins in both squid axoplasm and mammalian axons. Consistent with this, increased phosphorylation of NFs represents a well-documented feature early in the course of ALS. SDS-PAGE analysis of squid NFs reveals the presences of 60 kDa, 220 kDa (NF 220), and high molecular weight aggregates (HMW) composed of NF220. A marked increase in the phosphorylation of NF220 and HMW was observed in axoplasms perfused with G93A-SOD1, compared to WT-SOD1 perfused ones. Quantitative PHOSPHORIMAGER scanning revealed a 2-fold (p≦0.0284) increase in overall NF220 phosphorylation (n=4). These results showed that, in axons, fALS-related mutant SOD1 polypeptides promote an increase in NF phosphorylation through activation of one or more NF kinases.

### Example 3

### p38 Mediates the Inhibition of FAT Induced by Pathogenic SOD1

Vesicle motility assays showed mutant SOD1 polypeptides selectively inhibited anterograde FAT. Increased phosphorylation of neurofilament indicated altered kinase activity with mutant SOD1 and kinases represent a major mechanism for the regulation of conventional kinesin (Donelan, et al. (2002) J. Biol. Chem. 277:24232-42; Morfini, et al. (2006) Nat. Neurosci. 9:907-16; Morfini, et al. (2002) EMBO J. 23:281-93; Morfini, et al. (2001) Dev. Neurosci. 23:364-76). Accordingly, it was determined whether specific protein kinases mediated the effect of mutant SOD1 on anterograde FAT. To this end, axoplasms were co-perfused with recombinant G93A-SOD1 and specific pharmacological or peptide inhibitors of protein kinases. Co-perfusion approaches have facilitated the identification of kinase-dependent pathogenic pathways in other models. For example, the selective inhibition of anterograde FAT induced by filamentous forms of tau is prevented by co-perfusion with GSK3 inhibitors (Lapointe, et al. (2009) J. Neurosci. Res. 87(2):440-51), whereas the effects of pathogenic androgen receptor and Huntingtin on FAT are prevented by co-perfusion with JNK inhibitors (Morfini, et al. (2006) supra).

Co-perfusion of G93A-SOD1 with either CREBp (a peptide substrate that acts as a competitive inhibitor of GSK3) or with SP600125 (a specific inhibitor of JNK kinases (Morfini, et al. (2006) supra)) failed to block the inhibition of anterograde FAT by SOD1. In contrast, another pharmacological inhibitor, SB203580, completely prevented the effects of G93A-SOD1 on FAT. SB203580 is a highly specific pharmacological kinase inhibitor of the mitogen-activated protein kinase (MAPK) p38, and its specificity has been evaluated for more than 100 kinases (Fabian, et al. (2005) Nat. Biotechnol. 23:329-36). Similar results were obtained using AKA-5-035B, a specific pharmacological inhibitor of p38 structurally unrelated to SB203580 (Munoz, et al. (2007) J. Neuroinflammation 4:21). An inactive analog of AKA-5-035B (AKA-5-035B), failed to prevent the effects of G93A-SOD1 on anterograde FAT. The results of this analysis indicated that anterograde FAT rates for axoplasms co-perfused with G93A-SOD1 and either CREBp, SP600125, or AKA-5-035B were significantly different from those of axoplasm perfused with WT-SOD1, but FAT rates for axoplasms co-perfused with G93A-SOD1 and either SB203580 or AKA-5-035B were undistinguishable (FIG. 2). Taken together, these data indicated that inhibition of anterograde FAT by fALS-related SOD1 mutants depends upon activation of axonal p38. The ability of p38 inhibitors further indicated the inhibitory effects of mutant SOD1 proteins on FAT did not result from general alterations on axonal microtubules, deficits in ATP production, or steric interference of FAT by SOD1 aggregates.

To refine this identification, activation of various p38 and other NF kinases were evaluated in “sister” axoplasms. Activation-specific phosphoantibodies against GSK3, ERK and JNK did not reveal changes in the activity of these kinases between WT-SOD1 and G93A-SOD1-perfused axoplasms. In contrast, antibodies against phosphorylated, catalytically active p38 (p-p38) revealed a dramatic increase in p38 activation in axoplasms perfused with G93A-SOD1, compared to WT-SOD1-perfused ones. Similar results were observed in G85R-SOD1-perfused axoplasms. Quantitative analysis of immunoblots indicated G93A-SOD1 induced approximately a 4-fold increase in p38 activity, compared to WT-SOD1 perfused of p38 (n=8).

To evaluate the physiological relevance of these observations to fALS, NF phosphorylation was examined, as was the activation of p38 and other NF kinases in WT-SOD1 and G93A-SOD1 transgenic mice (Gurney, et al. (1994) supra). Spinal cord lysates were prepared from age-matched, presymptomatic (50 days of age) WT-SOD1 and G93A-SOD1 mice and processed for immunoblot analysis. Phosphorylation-dependent antibodies recognizing active forms of GSK3, JNK and ERK showed similar activation levels for these kinases, regardless of genotype. Consistent with results from isolated axoplasm, phosphorylation of p38 indicative of kinase activation was increased in spinal cord lysates of G93A-SOD1 as compared to those from WT-SOD1 mice. Similarly, SMI32 antibodies against a dephosphorylated NF heavy chain (NFH) epitope, showed a marked reduction in immunoreactivity in lysates derived from G93A-SOD1 relative to WT-SOD1 mice, indicating increased NFH phosphorylation in G93A-SOD1 mice. In contrast, phosphorylation-dependent SMI31 antibodies recognizing a different NFH epitope showed similar immunoreactivity in WT-SOD1 and G93A-SOD1 mice. Previous studies in isolated axoplasm have suggested that SMI32, but not SMI31, recognizes a NF epitope targeted by MAPKs. Taken together, results from this immunoblot analysis of isolated axoplasms and transgenic mice spinal cords, as well as co-perfusion experiments, all indicated that pathogenic, fALS-related forms of SOD1 activate the p38 pathway. Because neuronal cell bodies are absent from isolated axoplasms, effects of pathogenic SOD1 on p38 activity were axon-autonomous, independent of changes in the neuronal cell body.

### Example 4

### Active p38α Mimics the Effect of Pathogenic SOD1 on FAT

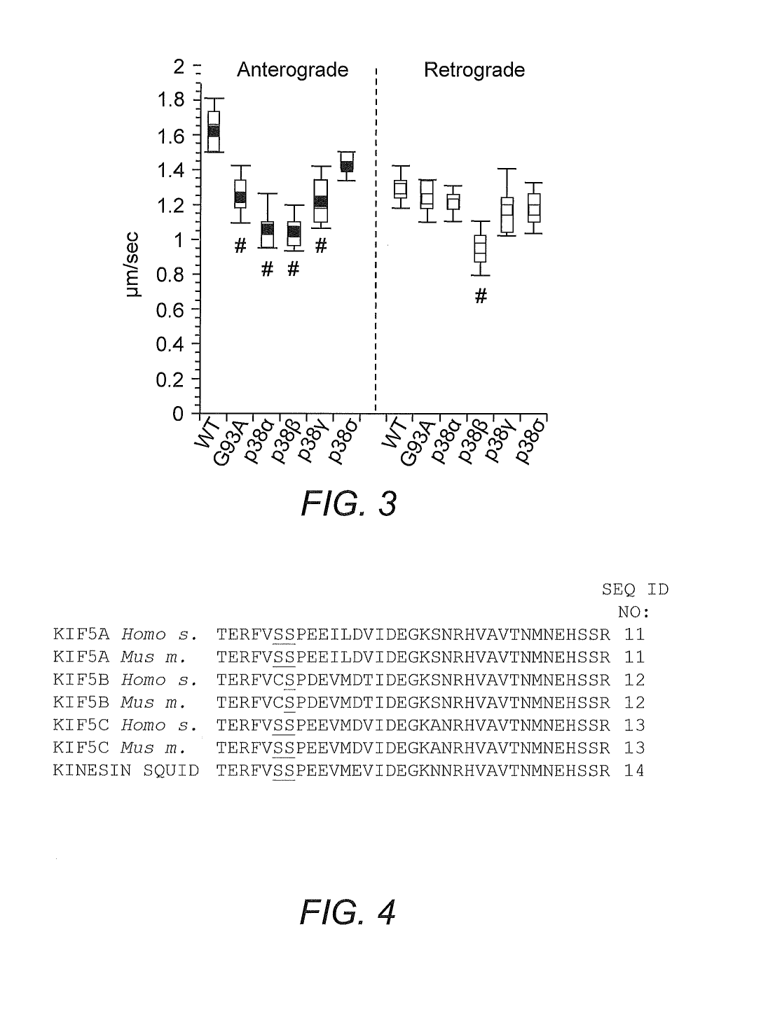

Pharmacological and biochemical experiments in isolated axoplasm indicated that pathogenic SOD1 mutants activate axonal p38, which in turn mediates their inhibitory effect on anterograde FAT. However, these experiments did not reveal the specific p38 isoform(s) involved. Four p38 genes exist in mammals (p38α, p38β, p38γ, and p38δ) (Kyriakis & Avruch (2001) Physiol. Rev. 81:807-69). The p38α and p38β kinases are major p38 isoforms within the nervous system (Lee, et al. (2000) J. Neurosci. Res. 60:623-31), as well as being the most prominent p38 isoforms expressed in motor neurons of the anterior horn spinal cord, the main cell type affected in ALS (Ackerley, et al. (2004) Mol. Cell. Neurosci. 26:354-64). The effect of specific p38 isoforms on FAT was evaluated using vesicle motility assays. The enzymatic activities of recombinant p38 isoforms were first normalized using in vitro kinase assays with ATF-2 as substrate. Perfusion of p38α (at 10 nM concentration) resulted in a selective inhibition of anterograde FAT that was indistinguishable from effects of pathogenic SOD1. Perfusion of 50 nM p3813 inhibited both anterograde and retrograde FAT. Perfusion of 100 nM p38γ had a more modest effect than 10 nM p38α, while perfusion of 50 nM of p385 did not affect FAT (FIG. 3). Quantitative analysis of these experiments showed that both p38α and G93A-SOD1-perfused axoplasms had similar effects in FAT. These results indicated that p38α mediates the inhibitory effects of pathogenic SOD1 on FAT.

### Example 5

### p38α Directly Phosphorylates Conventional Kinesin

Although p38α was implicated in mediating the inhibitory effect of pathogenic SOD1 on FAT, specific p38α targets relevant to these effects needed to be determined (Bendotti, et al. (2005) Neurodegener. Dis. 2:128-34). Previous analysis showed phosphorylation of kinesin-1 subunits by JNK3, a MAPK with similar substrate selectivity to p38α. These studies showed phosphorylation of kinesin-1 by JNK3 at a single serine residue impairs kinesin-1 translocation in vivo. These precedents prompted the determination of whether p38α could directly phosphorylate kinesin-1 subunits of conventional kinesin. In vitro kinase assays showed recombinant p38α phosphorylated a recombinant KHC construct encompassing the first 584 amino acid residues of kinesin-1c (KHC584).

Liquid chromatography tandem mass spectrometry (LC/MS/MS) was used to map KHC584 residues phosphorylated by p38α. To this end, in vitro phosphorylated KHC584 was directly digested with trypsin for LC/MS/MS analysis. Trypsin digest products were fractioned on a C18 HPLC connected to an on-line electrospray ion trap mass spectrometer. Peptide identification was done using SEQUEST algorithm. KHC584 identity was confirmed by identification of multiple kinesin-1C peptides. A peptide corresponding to amino acids 174-188 with unequivocal evidence of phosphorylation by p38α was identified based on spectrum analysis, cross correlation and delta correlation values. Tandem mass spectrometry analysis (MS/MS) by collision-induced dissociation further mapped phosphorylation on both Ser175 and Ser176, consistent with studies showing phosphorylation of Ser176 by the MAPK JNK3. Remarkably, serines 175 and 176 are conserved among squid, mouse and human kinesin-1s (FIG. 4). Taken together, results from these experiments identified kinesin-1 as a novel p38α substrate, and mapped the p38α acceptor residues to amino acids 175 and 176.

### Example 6

### Ser175-176 Phosphorylation Inhibits Kinesin-1 Function

Serines 175 and 176 are in a surface loop of the kinesin-1 motor domain, a region implicated in binding of kinesin-1 to microtubules (Sack, et al. (1997) Biochemistry 36:16155-65). Previous studies have shown that kinesin-1 translocation along axonal microtubules is impaired by phosphorylation of serine 176. Various GFP-tagged, truncated kinesin-1 constructs were expressed in cultured hippocampal neurons to evaluate effects of S175/176 phosphorylation on kinesin-1 motility in vivo. A GFP-tagged kinesin-1 construct encompassing the first 560 amino acids of kinesin-1 (KHC560-GFP-WT) selectively translocates and accumulates at the distal end of axons, but not dendrites (Jacobson, et al. (2006) Neuron 49:797-804). KHC560-GFP-WT translocation is so efficient that little or no fluorescence can be detected within cell bodies or along axons (Jacobson, et al. (2006) supra). To evaluate whether phosphorylation of serines 175 and 176 affects kinesin-1 translocation efficiency, the localization of KHC560-GFP-WT was compared with that of the phosphorylation-mimicking construct KHC560-GFP-S175E/S176E and its unphosphorylatable counterpart KHC560-GFP-S175A/S176A using quantitative fluorescence microscopy. Much less phosphomimetic KHC560-GFP-S175E/S176E construct accumulated at axonal tips than the KHC560-GFP-WT construct (56±2% vs. 87±3%, respectively, mean±SEM; T-test, p<0.0001), with more KHC560-GFP-S175E/S176E fluorescence in cell bodies and faint staining of neurites, the expected pattern if a significant fraction of phosphomimetic KHC560 were distributed like a soluble protein. Slightly less KHC560-GFP-S175A/S176A construct accumulated at axon tips compared to KHC560-GFP-WT, but accumulations were still significantly higher than KHC560-GFP-S175E/S176E (t-test, p<0.0001). Thus, a mutation of Ser175/176 mimicking phosphorylation reduces the efficiency of kinesin-1 translocation along microtubules in cultured neurons.

### Example 7

### p38 Activation by Mutant SOD1 Affects Neuronal Cell Viability

Multiple studies have documented activation of apoptotic mechanism in association with mutant SOD1 expression (Pasinelli, et al. (2004) Neuron 43:19-30). Results from experiments herein indicated that mutant SOD1-induced increases in p38 activity may be relevant not only to FAT, but also to cell viability. To evaluate the role of p38 in this process, cell viability (LDH) and caspase activity assays were performed using N2A cells stably transfected with WT-SOD1 or G85R-SOD1 constructs. Previous studies showed that exposure of N2A cells to low levels of cyclosporine (CsA) selectively increases apoptotic cell death in N2A cells expressing G85R-SOD1, compared to WT-SOD1-expressing ones (Maxwell, et al. (2004) Proc. Natl. Acad. Sci. USA 101:3178-83). N2A cells were exposed to various concentrations of CsA in the presence or absence of the p38 inhibitor SB203580. Significantly, results from both LDH and caspase-3 activity assays showed significant attenuation of cell death and caspase activation in G85R-SOD1 cells, indicating that apoptosis associated with mutant SOD1 is at least partially dependent on p38 activation.

### Example 8

### Mutant SOD1 Activates p38 Through an MKKK-Dependent Signaling Pathway

The activities of p38s are regulated by phosphorylation in vivo (Raman, et al. (2007) Oncogene 26(22):3100). p38s are substrates for MAPK kinases (MKKs), dual-specificity kinases that phosphorylate p38 on both a threonine and a tyrosine residue located at the activation loop of their catalytic domain (Lawler, et al. (1998) Curr. Biol. 8(25):1387). Dual phosphorylation of p38 at these residues is absolutely required for their activation. Four different MKK genes have been identified (MKK3, 4, 6 and 7; Raman, et al. (2007) supra). MKK3 and MKK6 are the major upstream activators of p38 (Raman, et al. (2007) supra). MKKs are also activated by phosphorylation within their activation loops, and this event is catalyzed by a group of serine/threonine kinases known as MAPK kinase kinases (MKKKs). Members of several MKKK families can activate the p38 pathway, including mixed-lineage kinases (MLKs 1, 2, 3, and 7), MEK kinases (MEKKs 1 to 4), apoptosis-inducing kinase (ASKs 1 and 2), transforming-growth factor beta (TGFβ)-activated kinase 1 (TAK1) and TAO (Thousand And One amino acids kinases 1 to 3) (Raman, et al. (2007) supra; Kumar, et al. (2003) Nat. Rev. Drug Discov. 2(9):717). The primary sequence in the activation loop of MAPKs alone does not specify their recognition by upstream protein kinases (Raman, et al. (2007) supra). Rather, elements distant from the activation loop determine recognition and binding. Multiple regions on MAPKs mediate specificity of interactions with substrates and regulators. For example, all MAPKs, including p38, have a cluster of negatively charged amino acids C-terminal to the kinase domain sequence. This site is referred to as the common docking (CD) domain, which interacts with the docking or D motif present in the amino terminus of its upstream MKK. Underscoring the importance of these domains, mutations of the acidic residues in the CD region not only decrease binding, but also diminish the efficiency of p38 activation or inhibition (Enslen, et al. (2000) EMBO J. 19(6):1301). Also, splice variants of MKK3 and MKK6 lacking the D domain site are less efficient in activating p38 (Raman, et al. (2007) supra). The strength of the interactions, based on the individual affinities and the number of interaction sites, plays a role in determining the cellular outcome of activation of multiple MAPK cascades that converge on a single protein.

In addition to the docking interactions of MAPKs with MKKs, substrates and regulators there are specific motifs that control the interactions of MKKKs and MKKs. A docking site termed DVD (domain for versatile docking) is found in several MKKs including MKK3 and MKK6, which are involved in p38 regulation. The DVD site is near the extreme C-terminus of the MKK and was shown to be involved in binding of MKKs with specific MKKKs including MEKK1, MEKK4, ASK1, TAO2 and TAK1 (all of which are able to activate the p38 pathway), but which is not involved with MLKs. The N lobe within the kinase domain of the MKKK was shown to bind to the MKK DVD site. Observations of p38 activation in an ALS mouse model prompted the evaluation of the role of MKKs in mediating the inhibitory effect of mutant SOD1 on FAT. To this end, pharmacological inhibitors of MKKKs were co-perfused along with SOD1-G93A in squid axoplasm. The MLK-specific inhibitor CEP11004 (Ganguly, et al. (2004) J. Neurochem. 88(2):469) was ineffective, but co-perfusion of mutant SOD1 with DVD peptide prevented the effects of mutant SOD1 on FAT. The DVD site on MKKs is required for docking and activation by a subset of upstream MKKKs (ASK1, TAK1, etc.), and peptides based on this sequences prevents MAPK activation in cultured cells (Takekawa, et al. (2005) Mol. Cell. 18(3):295). These data indicate that mutant SOD1 inhibition of FAT is mediated by an interaction between a MAPKKK and a MKK. Thus, this analysis indicates that mutant SOD1 proteins inhibit FAT through an MKKK-MKK-dependent, p38-specific signaling pathway.

What is claimed is:

1. A method for preserving fast axonal transport comprising contacting a cell that expresses a pathogenic superoxide dismutase 1 polypeptide with an effective amount of an agent that inhibits p38α activity thereby preserving fast axonal transport in the cell.

2. A method for preventing or treating Amyotrophic Lateral Sclerosis comprising administering to a subject in need of treatment an effective amount of an agent that inhibits p38α activity thereby preventing treating the subject's Amyotrophic Lateral Sclerosis.

3. A method for identifying an agent for treating Amyotrophic Lateral Sclerosis comprising contacting p38α with a test agent in the presence of kinesin-1 or a neurofilament heavy chain subunit, or a substrate fragment thereof, and determining whether the test agent inhibits the phosphorylation of the kinesin-1, neurofilament heavy chain subunit, or substrate fragment by the p38α thereby identifying an agent for treating Amyotrophic Lateral Sclerosis.

4. A method for monitoring treatment of Amyotrophic Lateral Sclerosis comprising determining, in a biological sample from a subject receiving therapy for Amyotrophic Lateral Sclerosis, the phosphorylation state of p38α, a neurofilament heavy chain subunit, or kinesin-1, wherein a decrease in the phosphorylation of p38α, neurofilament heavy chain subunit, or kinesin-1 after receiving therapy is indicative of treatment of the Amyotrophic Lateral Sclerosis.


SEQUENCE LISTING (14 sequences)

SEQ ID NO: 1 - DNA, 42 bp, Artificial sequence
  Synthetic oligonucleotide
gcgtaatacgactcactataggcactaggtggtacagggctc

SEQ ID NO: 2 - DNA, 43 bp, Artificial sequence
  Synthetic oligonucleotide
gcgtaatacgactcactataggcaggactccatctcttcttgg

SEQ ID NO: 3 - protein, 1032 aa, Homo sapiens
MAETNNECSIKVLCRFRPLNQAEILRGDKFIPIFQGDDSVVIGGKPYVFDRVFPPNTTQEQVYHACAMQIVKDVLAGYNGTIFAYGQTSSGKTHTMEGKLHDPQLMGIIPRIARDIFNHIYSMDENLEFHIKVSYFEIYLDKIRDLLDVTKTNLSVHEDKNRVPFVKGCTERFVSSPEEILDVIDEGKSNRHVAVTNMNEHSSRSHSIFLINIKQENMETEQKLSGKLYLVDLAGSEKVSKTGAEGAVLDEAKNINKSLSALGNVISALAEGTKSYVPYRDSKMTRILQDSLGGNCRTTMFICCSPSSYNDAETKSTLMFGQRAKTIKNTASVNLELTAEQWKKKYEKEKEKTKAQKETIAKLEAELSRWRNGENVPETERLAGEEAALGAELCEETPVNDNSSIVVRIAPEERQKYEEEIRRLYKQLDDKDDEINQQSQLIEKLKQQMLDQEELLVSTRGDNEKVQRELSHLQSENDAAKDEVKEVLQALEELAVNYDQKSQEVEEKSQQNQLLVDELSQKVATMLSLESELQRLQEVSGHQRKRIAEVLNGLMKDLSEFSVIVGNGEIKLPVEISGAIEEEFTVARLYISKIKSEVKSVVKRCRQLENLQVECHRKMEVTGRELSSCQLLISQHEAKIRSLTEYMQSVELKKRHLEESYDSLSDELAKLQAQETVHEVALKDKEPDTQDADEVKKALELQMESHREAHHRQLARLRDEINEKQKTIDELKDLNQKLQLELEKLQADYEKLKSEEHEKSTKLQELTFLYERHEQSKQDLKGLEETVARELQTLHNLRKLFVQDVTTRVKKSAEMEPEDSGGIHSQKQKISFLENNLEQLTKVHKQLVRDNADLRCELPKLEKRLRATAERVKALEGALKEAKEGAMKDKRRYQQEVDRIKEAVRYKSSGKRGHSAQIAKPVRPGHYPASSPTNPYGTRSPECISYTNSLFQNYQNLYLQATPSSTSDMYFANSCTSSGATSSGGPLASYQKANMDNGNATDINDNRSDLPCGYEAEDQAKLFPLHQETAAS

SEQ ID NO: 4 - protein, 1027 aa, Mus musculus
MAETNNECSIKVLCRFRPLNQAEILRGDKFIPIFQGDDSVIIGGKPYVFDRVFPPNTTQEQVYHACAMQIVKDVLAGYNGTIFAYGQTSSGKTHTMEGKLHDPQLMGIIPRIARDIFNHIYSMDENLEFHIKVSYFEIYLDKIRDLLDVTKTNLSVHEDKNRVPFVKGCTERFVSSPEEILDVIDEGKSNRHVAVTNMNEHSSRSHSIFLINIKQENVETEQKLSGKLYLVDLAGSEKVSKTGAEGAVLDEAKNINKSLSALGNVISALAEGTKSYVPYRDSKMTRILQDSLGGNCRTTMFICCSPSSYNDAETKSTLMFGQRAKTIKNTASVNLELTAEQWKKKYEKEKEKTKAQKETIAKLEAELSRWRNGENVPETERLAGEDSALGAELCEETPVNDNSSIVVRIAPEERQKYEEEIRRLYKQLDDKDDEINQQSQLIEKLKQQMLDQEELLVSTRGDNEKVQRELSHLQSENDAAKDEVKEVLQALEELAVNYDQKSQEVEEKSQQNQLLVDELSQKVATMLSLESELQRLQEVSGHQRKRIAEVLNGLMRDLSEFSVIVGNGEIKLPVEISGAIEEEFTVARLYISKIKSEVKSVVKRCRQLENLQVECHRKMEVTGRELSSCQLLISQHEAKIRSLTEYMQTVELKKRHLEESYDSLSDELARLQAHETVHEVALKDKEPDTQDAEEVKKALELQMENHREAHHRQLARLRDEINEKQKTIDELKDLNQKLQLELEKLQADYERLKNEENEKSAKLQELTFLYERHEQSKQDLKGLEETVARELQTLHNLRKLFVQDVTTRVKKSAEMEPEDSGGIHSQKQKISFLENNLEQLTKVHKQLVRDNADLRCELPKLEKRLRATAERVKALEGALKEAKEGAMKDKRRYQQEVDRIKEAVRYKSSGKRGHSAQIAKPVRPGHYPASSPTNPYGTRSPECISYTNNLFQNYQNLHLQAAPSSTSDMYFASSGATSVAPLASYQKANMDNGNATDINDNRSDLPCGYEAEDQAKLFPLHQETAAS

SEQ ID NO: 5 - protein, 1027 aa, Rattus norvegicus
MAETNNECSIKVLCRFRPLNQAEILRGDKFIPIFQGDDSVIIGGKPYVFDRVFPPNTTQEQVYHACAMQIVKDVLAGYNGTIFAYGQTSSGKTHTMEGKLHDPQLMGIIPRIARDIFNHIYSMDENLEFHIKVSYFEIYLDKIRDLLDVTKTNLSVHEDKNRVPFVRGCTERFVSSPEEILDVIDEGKSNRHVAVTNMNEHSSRSHSIFLINIKQENIETEQKLSGKLYLADLAGSEKVSKTGAEGAVLDEAKNINKSLSALGNVISALAEGTKSYVPYRDSKMTRILQDSLGGNCRTTMFICCSPSSYNDAETKSTLMFGQRAKTIKNTASVNLELTAEQWKKKYEKEKEKTKAQKETIAKLEAELSRWRNGENVPETERLAGEDSALAAEICEETPVNDNSSIVVRIAPEERQKYEEEIRRLYKQLDDKDDEINQQSQLIEKLKQQMLDQEELLVSTRGDNEKVQRELSHLQSENDAAKEEVKEVLQALEELAVNYDQKSQEVEEKSQQNQLLVDELSQKVATMLSLESEPQRLQEVSGHQRKRIAEVLNGLMKDLSEFSVIVGNGEIKLPVEISGAIEEEFTVARLYISKIKSEVKSVVKRCRQLENLQVECHRKMEVTGRELSSCQLLISQHEAKIRSLTEYMQTVELKKRHLEESYDSLSDELAKLQAQETVHEVALKDKEPDTQDAEEVKKALELQMENHREAHHRQLARLRDEINEKQKTIDELKDLDQKLQLELEKLQADYERLKNEENEKSAKLQELTFLYERHEQSKQDLKGLEETVARELQTLHNLRKLFVQDVTTRVKKSAEMEPEDSGGIHSQKQKISFLENNLEQLTEVHKQLVRDNADLRCELPKLEKRLRATAERVKALEGALKEAKEGAMKDKRRYQQEVDRIKEAVRYKSSGKRGHSAQIAKPVRPGHYPASSPTNPYGTRSPECISYTNNLFQNYQNLHLQAAPSSTSDVYFASNGATSVAPLASYQKANTDNGNATDINDNRSDLPCGYEAEDPAKLFPLHQETAAS

SEQ ID NO: 6 - protein, 963 aa, Homo sapiens
MADLAECNIKVMCRFRPLNESEVNRGDKYIAKFQGEDTVVIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKILRNTIQWLENELNRWRNGETVPIDEQFDKEKANLEAFTVDKDITLTNDKPATAIGVIGNFTDAERRKCEEEIAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKQPEGTGMIDEEFTVARLYISKMKSEVKTMVKRCKQLESTQTESNKKMEENEKELAACQLRISQHEAKIKSLTEYLQNVEQKKRQLEESVDALSEELVQLRAQEKVHEMEKEHLNKVQTANEVKQAVEQQIQSHRETHQKQISSLRDEVEAKAKLITDLQDQNQKMMLEQERLRVEHEKLKATDQEKSRKLHELTVMQDRREQARQDLKGLEETVAKELQTLHNLRKLFVQDLATRVKKSAEIDSDDTGGSAAQKQKISFLENNLEQLTKVHKQLVRDNADLRCELPKLEKRLRATAERVKALESALKEAKENASRDRKRYQQEVDRIKEAVRSKNMARRGHSAQIAKPIRPGQHPAASPTHPSAIRGGGAFVQNSQPVAVRGGGGKQV

SEQ ID NO: 7 - protein, 963 aa, Mus musculus
MADPAECNIKVMCRFRPLNESEVNRGDKYVAKFQGEDTVMIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKTLRNTIQWLENELNRWRNGETVPIDEQFDKEKANLEAFTADKDIAITSDKPAAAVGMAGSFTDAERRKCEEELAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKQPEGTGMIDEEFTVARLYISKMKSEVKTMVKRCKQLESTQTESNKKMEENEKELAACQLRISQHEAKIKSLTEYLQNVEQKKRQLEESVDSLGEELVQLRAQEKVHEMEKEHLNKVQTANEVKQAVEQQIQSHRETHQKQISSLRDEVEAKEKLITDLQDQNQKMVLEQERLRVEHERLKATDQEKSRKLHELTVMQDRREQARQDLKGLEETVAKELQTLHNLRKLFVQDLATRVKKSAEVDSDDTGGSAAQKQKISFLENNLEQLTKVHKQLVRDNADLRCELPKLEKRLRATAERVKALESALKEAKENASRDRKRYQQEVDRIKEAVRSKNMARRGHSAQIAKPIRPGQHPAASPTHPGTVRGGGSFVQNNQPVGLRGGGGKQS

SEQ ID NO: 8 - protein, 963 aa, Rattus norvegicus
MADPAECNIKVMCRFRPLNESEVNRGDKYVAKFQGEDTVMIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKTLRNTIQWLENELNRWRNGETVPIDEQFDKEKANLEAFTADKDVAITNDKPAAAIGMAGSFTDAERRKCEEEIAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKQPEGTGMIDEEFTVARLYISKMKSEVKTMVKRCKQLESTQTESNKKMEENEKELAACQLRISQHEAKIKSLTEYLQNVEQKKRQLEESVDSLGEELVQLRAQEKVHEMEKEHLNKVQTANEVKQAVEQQIQSHRETHQKQISSLRDEVEAKEKLITDLQDQNQKMVLEQERLRVEHERLKAVDQEKSRKLHELTVMQDRREQARQDLKGLEETVAKELQTLHNLRKLFVQDLATRVKKSAEVDSDDTGGSAAQKQKISFLENNLEQLTKVHKQLVRDNADLRCELPKLEKRLRATAERVKALESALKEAKENASRDRKRYQQEVDRIKEAVRSKNMARRGHSAQIAKPIRPGQHPAASPTHPGAVRGGGSFVQNNQPVGLRGGGGKQA

SEQ ID NO: 9 - protein, 957 aa, Homo sapiens
MADPAECSIKVMCRFRPLNEAEILRGDKFIPKFKGDETVVIGQGKPYVFDRVLPPNTTQEQVYNACAKQIVKDVLEGYNGTIFAYGQTSSGKTHTMEGKLHDPQLMGIIPRIAHDIFDHIYSMDENLEFHIKVSYFEIYLDKIRDLLDVSKTNLAVHEDKNRVPYVKGCTERFVSSPEEVMDVIDEGKANRHVAVTNMNEHSSRSHSIFLINIKQENVETEKKLSGKLYLVDLAGSEKVSKTGAEGAVLDEAKNINKSLSALGNVISALAEGTKTHVPYRDSKMTRILQDSLGGNCRTTIVICCSPSVFNEAETKSTLMFGQRAKTIKNTVSVNLELTAEEWKKKYEKEKEKNKTLKNVIQHLEMELNRWRNGEAVPEDEQISAKDQKNLEPCDNTPIIDNIAPVVAGISTEEKEKYDEEISSLYRQLDDKDDEINQQSQLAEKLKQQMLDQDELLASTRRDYEKIQEELTRLQIENEAAKDEVKEVLQALEELAVNYDQKSQEVEDKTRANEQLTDELAQKTTTLTTTQRELSQLQELSNHQKKRATEILNLLLKDLGEIGGIIGTNDVKTLADVNGVIEEEFTMARLYISKMKSEVKSLVNRSKQLESAQMDSNRKMNASERELAACQLLISQHEAKIKSLTDYMQNMEQKRRQLEESQDSLSEELAKLRAQEKMHEVSFQDKEKEHLTRLQDAEEMKKALEQQMESHREAHQKQLSRLRDEIEEKQKIIDEIRDLNQKLQLEQEKLSSDYNKLKIEDQEREMKLEKLLLLNDKREQAREDLKGLEETVSRELQTLHNLRKLFVQDLTTRVKKSVELDNDDGGGSAAQKQKISFLENNLEQLTKVHKQLVRDNADLRCELPKLEKRLRATAERVKALESALKEAKENAMRDRKRYQQEVDRIKEAVRAKNMARRAHSAQIAKPIRPGHYPASSPTAVHAIRGGGGSSSNSTHYQK

SEQ ID NO: 10 - protein, 956 aa, Mus musculus
MADPAECSIKVMCRFRPLNEAEILRGDKFIPKFKGEETVVIGQGKPYVFDRVLPPNTTQEQVYNACAKQIVKDVLEGYNGTIFAYGQTSSGKTHTMEGKLHDPQLMGIIPRIAHDIFDHIYSMDENLEFHIKVSYFEIYLDKIRDLLDVSKTNLAVHEDKNRVPYVKGCTERFVSSPEEVMDVIDEGKANRHVAVTNMNEHSSRSHSIFLINIKQENVETEKKLSGKLYLVDLAGSEKVSKTGAEGAVLDEAKNINKSLSALGNVISALAEGTKTHVPYRDSKMTRILQDSLGGNCRTTIVICCSPSVFNEAETKSTLMFGQRAKTIKNTVSVNLELTAEEWKKKYEKEKEKNKALKSVLQHLEMELNRWRNGEAVPEDEQISAKDQKSLEPCDNTPIIDNITPVVDGISAEKEKYDEEITSLYRQLDDKDDEINQQSQLAEKLKQQMLDQDELLASTRRDYEKIQEELTRLQIENEAAKDEVKEVLQALEELAVNYDQKSQEVEDKTRANEQLTDELAQKTTTLTTTQRELSQLQELSNHQKKRATEILNLLLKDLGEIGGIIGTNDVKTLADVNGVIEEEFTMARLYISKMKSEVKSLVNRSKQLESAQMDSNRKMNASERELAACQLLISQHEAKIKSLTDYMQNMEQKRRQLEESQDSLSEELAKLRAQEKMHEVSFQDKEKEHLTRLQDAEEVKKALEQQMESHREAHQKQLSRLRDEIEEKQRIIDEIRDLNQKLQLEQERLSSDYNKLKIEDQEREVKLEKLLLLNDKREQAREDLKGLEETVSRELQTLHNLRKLFVQDLTTRVKKSVELDSDDGGGSAAQKQKISFLENNLEQLTKVHKQLVRDNADLRCELPKLEKRLRATAERVKALESALKEAKENAMRDRKRYQQEVDRIKEAVRAKNMARRAHSAQIAKPIRPGHYPASSPTAVHAVRGGGGGSSNSTHYQK

SEQ ID NO: 11 - protein, 35 aa, Homo sapiens
TERFVSSPEEILDVIDEGKSNRHVAVTNMNEHSSR

SEQ ID NO: 12 - protein, 35 aa, Homo sapiens
TERFVCSPDEVMDTIDEGKSNRHVAVTNMNEHSSR

SEQ ID NO: 13 - protein, 35 aa, Homo sapiens
TERFVSSPEEVMDVIDEGKANRHVAVTNMNEHSSR

SEQ ID NO: 14 - protein, 35 aa, Loligo pealeii
TERFVSSPEEVMEVIDEGKNNRHVAVTNMNEHSSR